(19) United States
(12) Patent Application Publication
(10) Pub. No.: US 2014/0098943 A1
(43) Pub. Date: Apr. 10, 2014

(54) MOBILE X-RAY IMAGING APPARATUS

(71) Applicant: CANON KABUSHIKI KAISHA, TOKYO (JP)

(72) Inventors: Satoru Omura, Chigasaki-shi (JP); Naofumi Sekine, Kawasaki-shi (JP)

(73) Assignee: CANON KABUSHIKI KAISHA, TOKYO (JP)

(21) Appl. No.: 14/044,056

(22) Filed: Oct. 2, 2013

(30) Foreign Application Priority Data
Oct. 5, 2012 (JP) 2012-223563

(51) Int. Cl. A61B 6/00

(52) U.S. Cl. CPC A61B 6/4405
USPC 378/198

(57) ABSTRACT

A mobile X-ray imaging apparatus includes an X-ray tube configured to emit X-rays, an extendable arm configured to support the X-ray tube, a support post configured to move the extendable arm in a direction perpendicular to the ground, a carriage portion configured to control the X-ray tube to emit the X-rays and to support the support post, and including a movable unit, a monitor disposed on an upper surface of the carriage portion, and an arm opening and closing unit configured to open and close the extendable arm with respect to the support post.

### BACKGROUND OF THE INVENTION

1. Field of the Invention

The present invention relates to radiation imaging apparatuses for obtaining a radiation image from radiation transmitted through a subject, and more particularly, the present invention relates to mobile X-ray imaging apparatuses.

2. Description of the Related Art

In recent years, as medical X-ray imaging apparatuses, mobile X-ray imaging systems have been prevalently used. Mobile X-ray imaging systems are generally used for performing X-ray photography in a hospital room or an operating room. Mobile X-ray imaging apparatuses typically include an X-ray tube for emitting X-rays, an X-ray detector for detecting the X-rays transmitted through a body part of a patient, and a C-shaped arm for holding the X-ray tube on one end and the X-ray detector at the other end.

In performing the X-ray photography using the mobile X-ray imaging system, to position the X-ray tube over a subject lying on the bed, a means for changing positions over the bed needs to be provided for the X-ray tube. Especially, in a case where the arms and legs of the subject are to be photographed, the positioning of the X-ray detector and the X-ray tube is not maintained in a good condition if it is not possible to locate the X-ray tube at any position over the bed. In such a case, appropriate images are not provided.

Further, when the mobile X-ray imaging system is moved in a narrow space between beds in a hospital room or moved in a corridor in a hospital ward where stretchers and various medical devices come and go, for movement, it is desirable that the entire size of the system be reduced to a compact size.

For that purpose, an arm for supporting the X-ray tube may be provided with a mechanism for extending the arm during X-ray photography, and contracting it during movement. For example, in an exemplary embodiment in Japanese Patent Application Laid-Open No. 2006-81690, an extendable arm for supporting an X-ray tube is provided to extend the arm for X-ray photography and to shorten the arm for storage in movement.

In operator's round visit with the mobile X-ray imaging system, on a monitor disposed on a movable carriage portion, information and location of a patient to be photographed next is displayed and the operator can check the information while moving, and thereby smooth round visit can be performed. In known X-ray imaging systems like that discussed in Japanese Patent Application Laid-Open No. 2006-81690, however, the X-ray tube is disposed near the monitor when the system is moved, and this prevents the operator from checking the monitor during movement.

### SUMMARY OF THE INVENTION

Aspects of present invention are directed to mobile X-ray imaging apparatuses enabling an operator to check information on a monitor disposed even in a conventional way in moving the apparatus.

According to an aspect of the present invention, a mobile X-ray imaging apparatus includes an X-ray tube configured to emit X-rays, an extendable arm configured to support the X-ray tube, a support post configured to move the extendable arm in a direction perpendicular to the ground, a carriage portion configured to control the X-ray tube to emit the X-rays and to support the support post, and including a movable unit, a monitor disposed on an upper surface of the carriage portion, and an arm opening and closing unit configured to open and close the extendable arm with respect to the support post.

According to exemplary embodiments of the present invention, a mobile X-ray imaging apparatus that enables an operator to check information displayed on a monitor while moving the apparatus, and that increases the operator's ease of operability is disclosed.

Further features of the present invention will become apparent from the following description of exemplary embodiments with reference to the attached drawings.

### BRIEF DESCRIPTION OF THE DRAWINGS

FIGS. 1A and 1B illustrate a structure of a mobile X-ray imaging apparatus according to a first exemplary embodiment of the present invention.

FIGS. 2A and 2B illustrate an arm and a support post according to the first exemplary embodiment of the present invention.

FIG. 3 is a flowchart of a system according to the first exemplary embodiment of the present invention.

FIGS. 4A and 4B illustrate an arm and a support post according to a second exemplary embodiment of the present invention.

FIG. 5 is a flowchart of a system according to the second exemplary embodiment of the present invention.

FIGS. 6A and 6B illustrate a structure of a mobile X-ray imaging apparatus according to a third exemplary embodiment of the present invention.

FIG. 7 illustrates an arm and a support post according to the third exemplary embodiment of the present invention.

FIG. 8 illustrates a structure of a mobile X-ray imaging apparatus according to a fourth exemplary embodiment of the present invention.

FIG. 9 is a flowchart of a system according to the fourth exemplary embodiment of the present invention.

### DESCRIPTION OF THE EMBODIMENTS

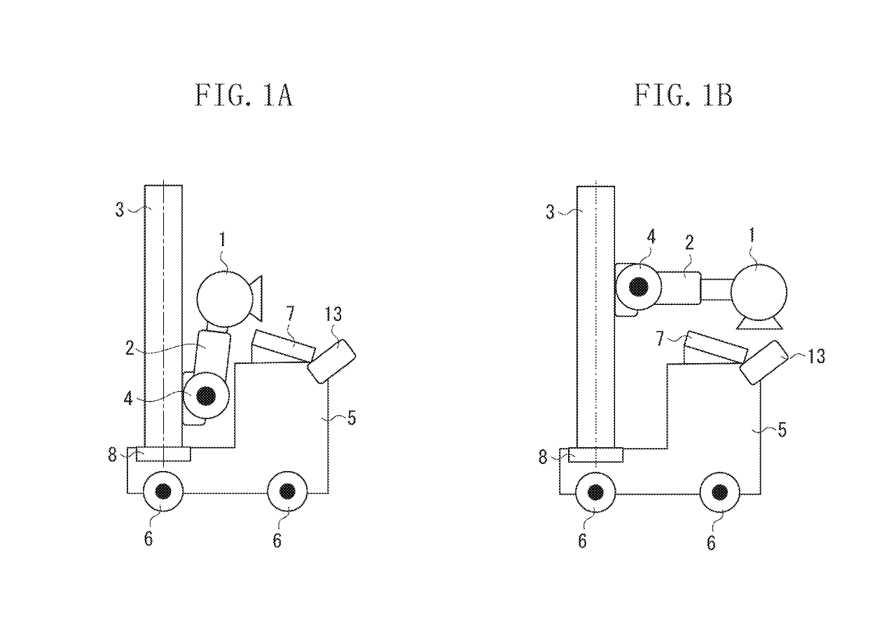

Hereinafter, a first exemplary embodiment will be described. FIGS. 1A and 1B illustrate a structure of a mobile X-ray imaging apparatus according to the first exemplary embodiment of the present invention.

FIG. 1A illustrates a moving state of the apparatus. FIG. 1B illustrates the mobile X-ray imaging apparatus when an arm of the apparatus has been extended in X-ray photography.

In FIG. 1A, an X-ray tube 1 emits X-rays. An arm 2 supports the X-ray tube 1, and has an extendable portion. A support post 3 supports the arm 2. An arm supporting part 4 has a portion (arm opening and closing portion) for connecting the arm 2 and the support post 3 and enabling the arm 2 to open and close with respect to the support post 3. A carriage portion 5 supports the support post 3. A moving mechanism 6 enables the carriage portion 5 to move. In the moving mechanism 6, a plurality of wheels or casters is arranged on the ground, and the wheels or the casters are rotated to move the carriage portion 5.

A monitor 7 is disposed on an upper surface of the carriage portion 5. The monitor 7 displays information of a patient and a location of the patient to be photographed in an operator's round visit. A support post rotating portion 8 connects the carriage portion 5 and the support post 3 to serve as a bearing. The support post rotating portion 8 enables the support post 3 to rotate around the axis perpendicular to the ground on which the wheels move, i.e., around the vertical axis, on the carriage portion 5. The support post rotating portion 8 also serves as a non-excitation electromagnetic brake, and in a state in which electric power is applied to the non-excitation electromagnetic brake, the rotation of the support post 3 can be stopped at a desired position. A handle 13 is provided in the carriage portion 5. The operator holds the handle 13 in moving the apparatus to control the moving direction of the apparatus. Such a structure prevents the X-ray tube 1 from being disposed over the monitor 7 when the apparatus is being moved, and enables the operator to check the information displayed on the monitor 7 while moving, and thereby the ease of operability is improved.

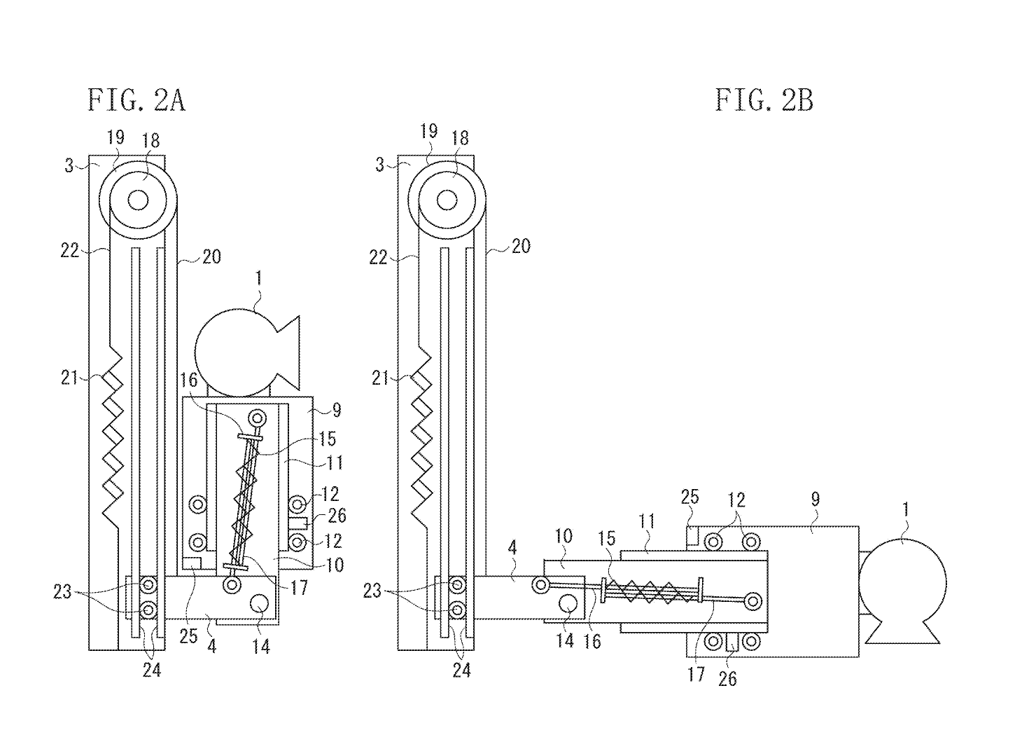

FIGS. 2A and 2B illustrate a specific structure of the arm 2 and the support post 3. FIG. 2A illustrates a storage state (non-operational state) of the arm 2 and the support post 3 when, for example, the apparatus is being moved or not operated in X-ray photography. FIG. 2B illustrates an extended state (operational state) of the arm and the support post when, for example, the arm is extended for X-ray photography.

In FIGS. 2A and 2B, a first arm 9 supports the X-ray tube 1. A guide plate 11 is fixed on the surface of a second arm 10, and the guide plate 11 has a linear shape in the length-wise direction of the second arm 10. Cam followers 12 are provided in the first arm 9, and rotate on the guide plate 11. The guide plate 11 and the cam followers 12 are similarly provided to the opposite surface of the second arm 10. To improve the slip of the cam followers 12, it is desirable to smooth the surface of the guide plate 11 as much as possible. Alternatively, the guide plate 11 may not be provided and the cam followers 12 may rotate on the surface of the second arm 10. Such structure enables the first arm 9 to move along the second arm 10 to extend and contract the arm 2. In the present exemplary embodiment, the two-stage extendable arm having the first and second arms has been described. Alternatively, an arm having two or more extendable stages may be employed, and in such a case, the guide plates 11 and the cam followers 12 are to be similarly provided for each extendable stage depending on the number of stages.

The arm supporting portion 4 connects the second arm 10 and the support post 3. A rotation portion 14 joins the second arm 10 and the arm supporting portion 4. The rotation of the rotation portion 14 rotates the second arm 10 with respect to the arm supporting portion 4. To reduce the width of the arm 2, it is desirable to use a gas spring as a compression spring 15. A first spring guide 16 is rotatable at one end on the arm supporting portion 4. A second spring guide 17 is rotatable at one end on the second arm 10. The compression spring 15 is compressed when the second arm 10 is extended from the storage state to the horizontal state (extended or operational state) as illustrated in FIGS. 2A and 2B. In such structure, the force to return the second arm 10 to the storage state acts, and this enables the operator to switch the arm from the storage state to the extended state and vice versa with a minimal force.

A pulley 18 is provided in an upper part in the support post 3. A tapered pulley 19 having a diameter larger than the pulley 18 is provided coaxially with the pulley 18. A first wire 20 is fixed to the arm supporting portion 4 at one end, and wound around the tapered pulley 19 at the other end. A second wire 22 is connected to an extension spring 21 at one end, and wound around the pulley 18 at the other end. The taper angle of the tapered pulley 19 is set to balance the force of the extension spring 21 to a weight of the members provided to the side from the arm supporting portion 4 to the X-ray tube 1 even if the length of the extension spring 21 is changed.

A cam followers 23 are provided on the arm supporting portion 4. Slide guide plates 24 are provided in the support post 3 to hold the cam followers 23. To improve the slip of the cam followers 23, it is desirable to smooth the surface of the slide guide plates 24 where the plates contact the cam followers 23 as much as possible. With such structure, the force of the extension spring 21 applies a predetermined small force to the members provided at the side from the arm supporting portion 4 to the X-ray tube 1, so that the arm 2 can be moved up and down along the support post 3 in the direction perpendicular to the ground. The cam followers 23 and the slide guide plates 24 enable the arm supporting portion 4 to move up and down along the support post 3 without changing the angle with the support post 3. In other words, the rotation of the arm 2 around the rotation portion 14 enables the arm 2 to change the opening and closing angle with respect to the support post 3.

An angle sensor 25 detects an angle of the second arm 10 to the ground. In the first exemplary embodiment, the angle sensor 25 detects whether the second arm 10 has been horizontally set. As the angle sensor 25, an acceleration sensor for detecting the ground direction is desirably employed. A lock portion 26 is used to lock the extension and contraction of the first arm 9 and the second arm 10. That is, the lock portion 26 serves as an arm extension and contraction lock unit for restricting the extension and contraction operation. As the lock portion 26, a permanent electromagnetic holder is desirably employed, and in the first arm 9, the lock portion 26 is provided to contact the surface of the second arm 10 of a magnetic substance. By the structure, depending on whether electric power is applied to the lock portion 26, the lock portion 26 attracts the second arm 10 to lock the extension and contraction of the first arm 9 and the second arm 10.

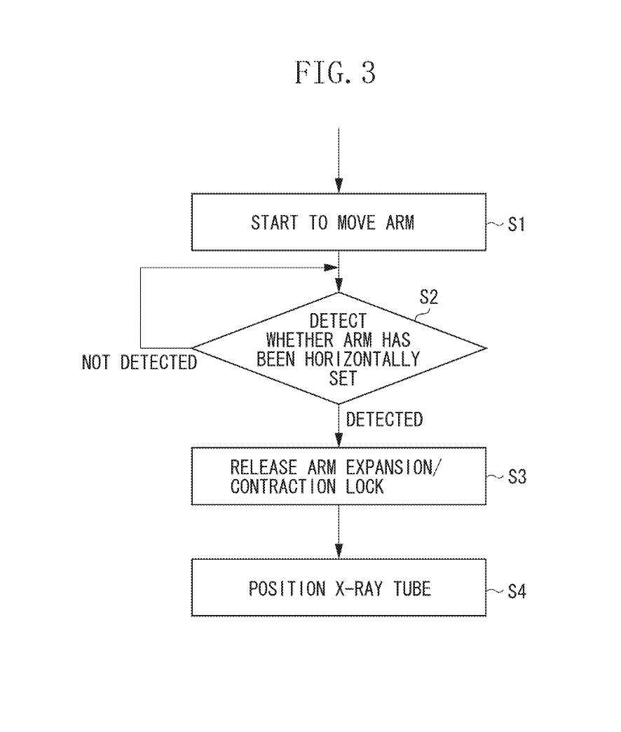

FIG. 3 is a flowchart of a system according to the first exemplary embodiment of the present invention. In FIG. 3, in step S1, the arm 2 starts to move in response to movement of the X-ray tube 1. In step S2, the arm 2 moves to open and close with respect to the support post 3, and it is detected whether the arm 2 has been set to the horizontal position. If the horizontal state of the arm 2 has not been detected (“NOT DETECTED” in step S2), the processing in step S2 is repeated. In step S2, if the horizontal state of the arm 2 has been detected (“DETECTED” in step S2), the process proceeds to step S3. In step S3, electric power is supplied to the lock portion 26 to release the extension and contraction lock of the arm 2. In step S4, positioning operation of the X-ray tube 1 is performed, and then, the process proceeds to an X-ray photography process.

With the above-described structure, the extension and contraction of the arm 2 is allowed only when the arm 2 has been set to the horizontal position. Thus, the operator can move the X-ray tube 1 while maintaining the height of the X-ray tube 1 at a certain height in the positioning operation of the X-ray tube 1. This increases the operability of the operator.

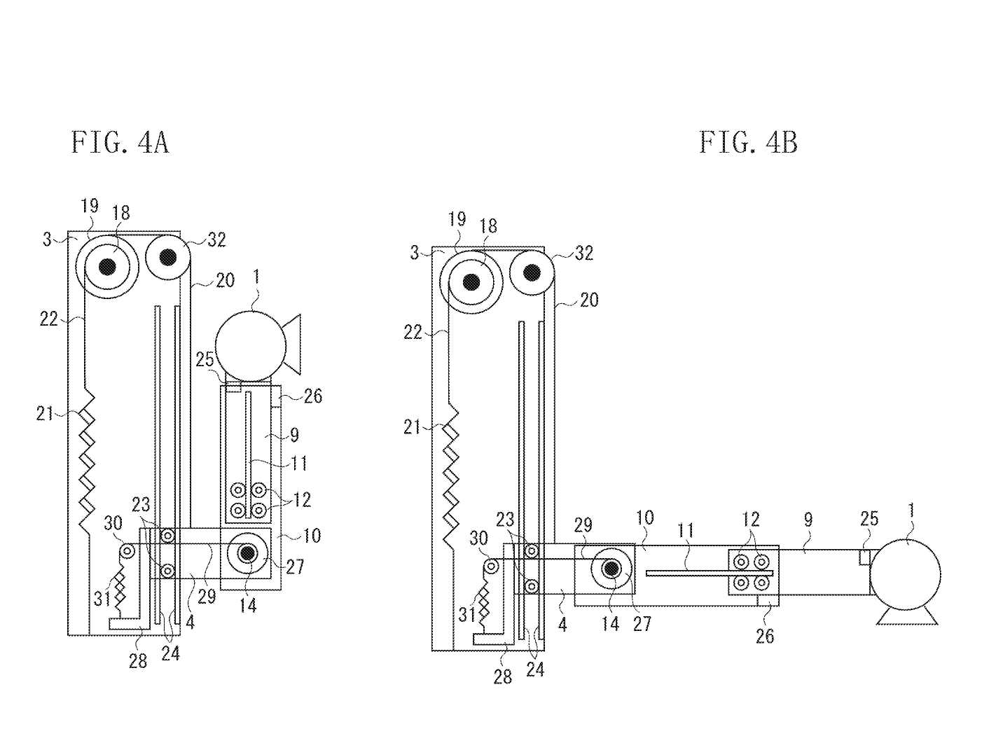

Hereinafter, a second exemplary embodiment is described. FIGS. 4A and 4B illustrate a structure of a mobile X-ray imaging apparatus according to the second exemplary embodiment of the present invention.

In the second exemplary embodiment, as compared to the first exemplary embodiment, the width of the arm is reduced, and the opening and closing operation of the arm 2 can be locked. Thus, an applicable range of exemplary embodiments of the present invention can be widened.

FIG. 4A illustrates a storage state of the arm and the support post when the apparatus is being moved. FIG. 4B illustrates an extended state of the arm and the support post when the arm is extended in X-ray photography. In FIGS. 4A and 4B, as described in the first exemplary embodiment, the extension and contraction mechanism of the first arm 9 and the second arm 10 can be implemented by the guide plates 11 and the cam followers 12. In the present exemplary embodiment, the two-stage extendable arm having the first and second arms has been described. Alternatively, an arm having two or more stages may be employed, and in such a case, the guide plates 11 and the cam followers 12 are to be similarly provided depending on the number of stages.

The arm supporting portion 4 connects the second arm 10 and the support post 3. The rotation portion 14 joins the second arm 10 and the arm supporting portion 4. The second arm 10 rotates around the rotation center of the rotation portion 14 with respect to the arm supporting portion 4. A tapered pulley 27 is provided coaxially with the rotation portion 14, and rotates in synchronization with the rotation of the rotation portion 14. To the rotation portion 14, a non-excitation electromagnetic brake is further provided. In a state electric power is being applied to the non-excitation electromagnetic brake, the rotation of the second arm 10 can be stopped at a position. In other words, the rotation portion 14 includes an arm opening and closing unit for fixing an opening and closing angle of the arm 2. The rotation brake in the rotation portion 14 is controlled by a signal from a brake input unit provided in the apparatus. In the second exemplary embodiment, the brake input unit is provided in the X-ray tube 1, or near the X-ray tube 1. To the rotation portion 14 according to the first exemplary embodiment, a rotation brake similar to that in the second exemplary embodiment may be provided.

A spring supporting portion 28 is fixed on the arm supporting portion 4. A wire 29 is wound around the tapered pulley 27 at one end. An extension spring 31 is fixed to the spring support portion 28 at one end, and connected to one end of the wire 29 at the other end to maintain the tension via a pulley 30. The taper angle of the tapered pulley 27 is set to balance the force of the extension spring 31 to the rotational moment of the arm 2 even if the length of the extension spring 31 is changed. As illustrated in FIGS. 4A and 4B, the tension spring 31 is pulled by extending the second arm 10 from the storage state to the horizontal state. In this structure, the force to return the second arm 10 to the storage state acts, and this enables the operator to switch the arm from the storage state to the extended state or in the opposite state with a small force.

As compared to the first exemplary embodiment, the internal structure of the support post 3 is widened by providing the extension spring 31. Due to the structure, the wire 20 can maintain the tension via a pulley 32. Except the above-described structure, the structure from the small-sized pulley 18 to the slide guide plates 24 is similar to that in the first exemplary embodiment. The structure enables the arm 2 to move up and down along the support post 3 in the direction perpendicular to the ground. The cam followers 23 and the slide guide plates 24 enable the arm supporting portion 4 to move up and down along the support post 3 without changing the angle with the support post 3. In other words, the rotation of the arm 2 around the rotation portion 14 enables the arm 2 to change the opening and closing angle with respect to the support post 3.

In the second exemplary embodiment, the angle sensor 25 detects whether the first arm 10 has been horizontally set. A lock portion 26 is used to lock the extension and contraction of the first arm 9 and the second arm 10. The lock portion 26 is disposed in the second arm 10 to contact the surface of the first arm 9 of a magnetic substance. By the structure, depending on whether electric power is applied to the lock portion 26, the lock portion attracts the first arm 9 to lock the extension and contraction of the first arm 9 and the second arm 10.

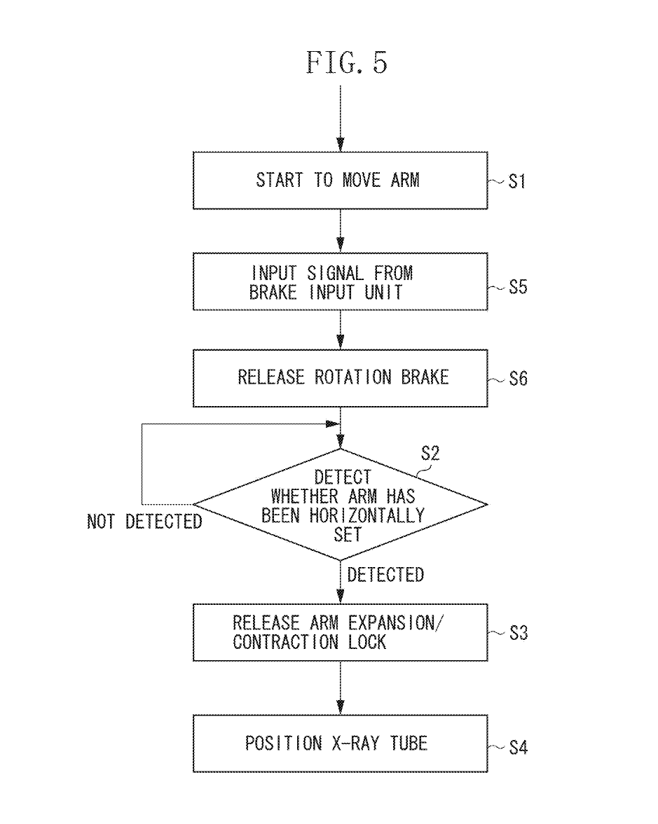

FIG. 5 is a flowchart of a system according to the second exemplary embodiment of the present invention. In FIG. 5, in step S5, a signal is input from the brake input unit provided in the X-ray tube 1. In step S6, by the signal input in step S5, the rotation brake of the rotation portion 14 is released, and this enables the arm 2 to rotate.

With the above-described structure, the arm can be moved upward only by the slide mechanism of the support post to move the X-ray tube 1 upward only in the vertical direction. By the structure, while the X-ray tube 1 is kept in the certain horizontal position, the height of the X-ray tube 1 can be changed. This increases the operability of the operator.

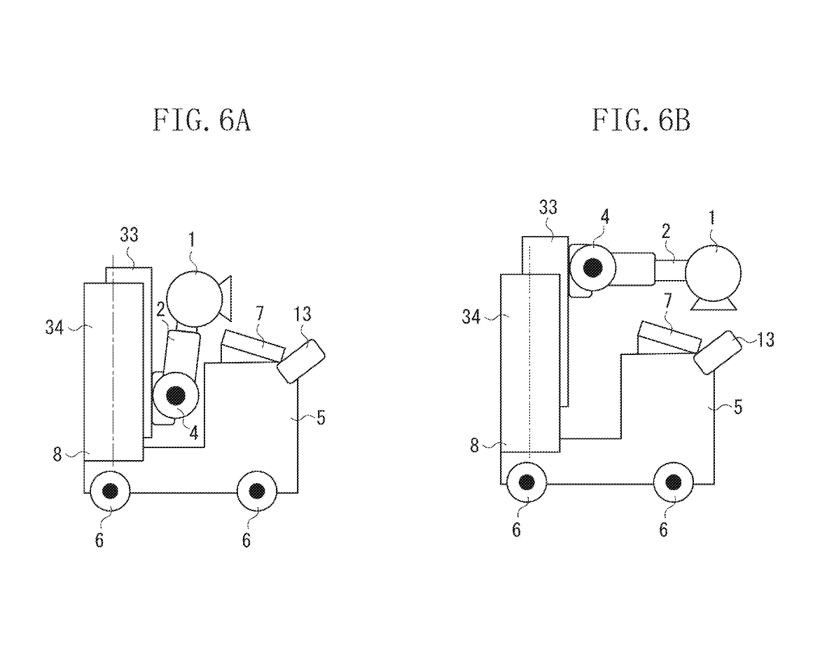

Hereinafter, a third exemplary embodiment is described. FIGS. 6A and 6B illustrate a structure of a mobile X-ray imaging apparatus according to the third exemplary embodiment of the present invention. In the third exemplary embodiment, as compared to the second exemplary embodiment, the support post 3 is configured to be extendable and thereby an applicable range of the exemplary embodiments of the present invention can be widened.

FIG. 6A illustrates a moving state of the apparatus. FIG. 6B illustrates the mobile X-ray imaging apparatus when the arm of the apparatus has been extended in X-ray photography. As compared to FIG. 1 according to the first exemplary embodiment, in FIG. 6, a first support post 33 for supporting the arm 2 is provided. The arm supporting portion 4 has a unit for connecting the arm 2 and the first support post 33 and enabling the arm 2 to open or close with respect to the first support post 33. A second support post 34 allows the first support post 33 to extend and contract with respect to the second support post 34. The carriage portion 5 supports the second support post 34. The support post rotating portion 8 connects the carriage portion 5 and the second support post 34 to serve as a bearing. The support post rotating portion 8 enables the second support post 34 to rotate around the axis perpendicular to the ground on the carriage portion 5. Further, in the support post rotating portion 8, a non-excitation electromagnetic brake is provided, and in a state electric power is being applied to the non-excitation electromagnetic brake, the rotation of the second support post 34 can be stopped at a position.

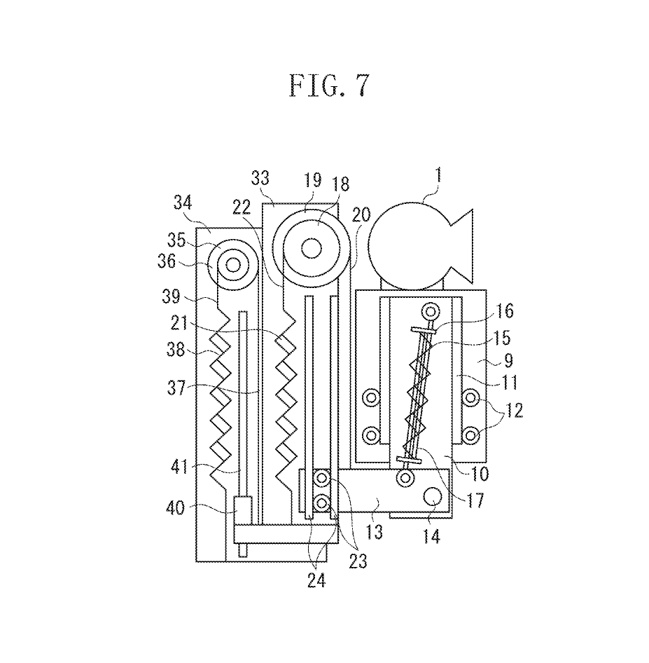

FIG. 7 illustrates a specific structure of the arm 2, the support post 33, and the support post 34. In FIG. 7, the structure of the arm 2 is similar to that described in the first exemplary embodiment. An internal structure of the second support post 34 is similar to the structure in the support post 3 described in the first exemplary embodiment.

A small-diameter pulley 35 is provided in an upper part in the second support post 34. A tapered pulley 36 is provided coaxially with the small-diameter pulley 35. A third wire 37 is fixed to a bottom part of the first support post 33 at one end, and wound around the tapered pulley 36 at the other end. A fourth wire 39 is connected to an extension spring 38 at one end, and wound around the small-diameter pulley 35 at the other end. The taper angle of the tapered pulley 36 is set to balance the force of the extension spring 38 to a weight of the members provided to the side from the first support post 33 to the X-ray tube 1 even if the length of the extension spring 38 is changed.

A linear guide 40 is provided in the bottom part of the first support post 33. A linear guide rail 41 is provided in the second support post 34 to guide the linear guide 40. With such structure, the extension spring 38 applies a predetermined small force to the members provided at the side from the first support post 33 to the X-ray tube 1, so that the first support post 33 can be moved up and down. With the linear guide 40 and the linear guide rail 41, the first support post 33 can be moved up and down along the second support post 34 in the direction perpendicular to the ground. In other words, a support post extension and contraction unit enabling the support posts 33 and 34 to extend and contract in the vertical direction can be provided.

With the above-described structure, the support posts can extend and contract in the vertical direction, which allows compact storage of the arm and support posts in the movement, including the arm extension and contraction. As a result, the front visibility of the operator in moving the apparatus can be increased. Consequently, the mobile X-ray imaging apparatus can be provided that enables the operator to easily grasp the environment while moving the apparatus, to check a next subject from the monitor information, and to move the apparatus smoothly.

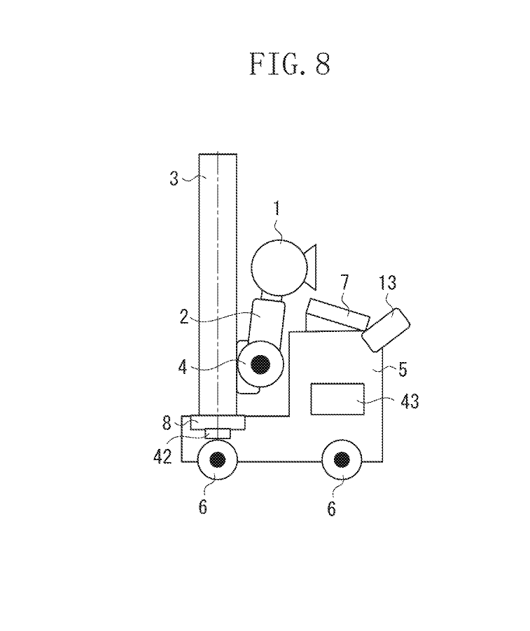

Hereinafter, a fourth exemplary embodiment is described. FIG. 8 illustrates a structure of a mobile X-ray imaging apparatus according to the fourth exemplary embodiment of the present invention. In the fourth exemplary embodiment, as compared to the second exemplary embodiment, by a rotational angle of the support post 3, opening and closing operation of the arm 2 is restricted, and thereby an applicable range of the present invention can be widened.

In FIG. 8, the X-ray tube 1 to the support post rotating portion 8, and the handle 13 are similar to those described in FIG. 1 according to the first exemplary embodiment. The arm supporting portion 4 is similar to that described in FIG. 4 according to the second exemplary embodiment. The rotation portion 14 of the arm supporting portion 4 is provided with the rotation brake. A support post rotation detection unit 42 detects a rotational angle of the support post rotating portion 8 to grasp a direction of the support post 3. As a structure of the support post rotation detection unit 42, it is desirable to employ a structure in which the bottom surface of the support post rotating portion 8 is divided into the north pole and the south pole by a horizontal line passing through the rotation axis of the post pole. Further, the structure of the support post rotation detection unit 42 is provided with a magnetic sensor to calculate a support post rotational angle from a change in a magnetic force due to rotation of the support post. In another structure, the support post rotation detection unit 42 may be provided with a variable resistor, in which electrical resistance varies depending on rotation of the support post 3 to measure the electrical resistance to calculate a support post rotational angle.

A control unit 43 controls the rotation brake of the rotation portion 14 based on a detection result of the support post rotation detection portion 42 to restrict opening and closing operation of the arm 2 depending on the direction of the support post 3. In the forth exemplary embodiment, as compared to the second exemplary embodiment, the control unit 43 also serves as the rotation brake input unit of the rotation portion 14. Further, the control unit includes a correspondence table of arm opening and closing angles and support post rotational angles for preventing the X-ray tube 1 and the arm 2 from interfering with the carriage portion 5 due to the shapes of the carriage portion 5, the X-ray tube 1, and the arm 2 when the arm 2 is to be set to the horizontal position.

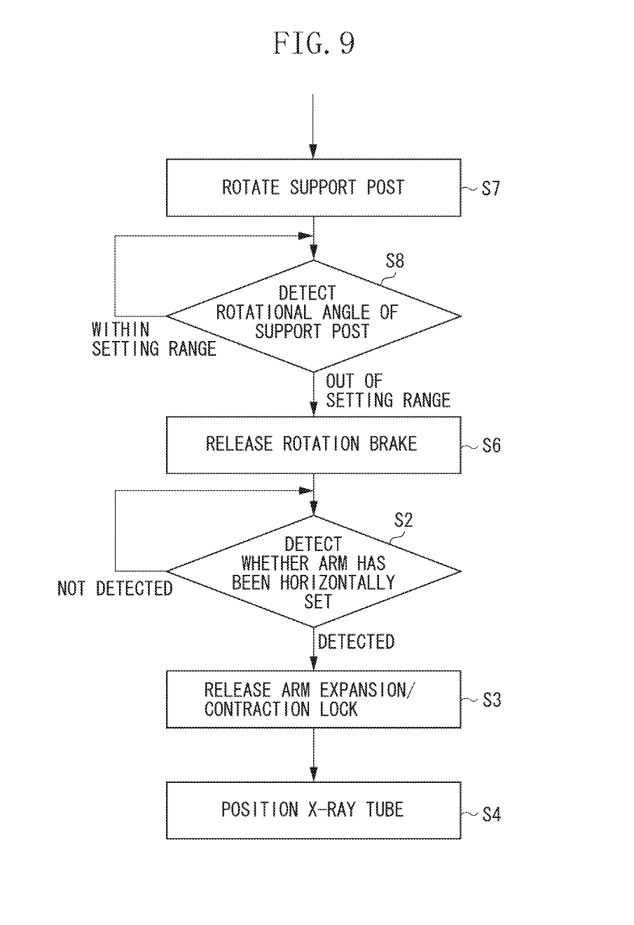

FIG. 9 is a flowchart of a system according to the fourth exemplary embodiment of the present invention. In FIG. 9, in step S7, the support post 3 is rotated to position the X-ray tube 1. In step S8, based on a detection result of the support post rotation detection unit 42, whether the angle is the support post rotational angle at which the arm 2 can be set to the horizontal position is calculated from the correspondence table. If the arm 2 cannot be set to the horizontal position at the angle (“WITHIN SETTING RANGE” ins step S8), the processing in step S8 is continued to perform the support post rotational angle detection. If the arm 2 can be set to the horizontal position at that support post rotational angle (“OUT OF SETTING RANGE” in step S8), the process proceeds to step S6. In step S6, the rotation brake of the rotation portion 14 is released to allow the arm 2 to open or close. In other words, within the range of support post rotational angles of the support post 3, the arm opening and closing lock unit is not released.

In the present exemplary embodiment, it is assumed that the arm 2 is to be set to the horizontal position, however, the arm 2 may be used in a state other than the horizontal state. In such a case, the angle sensor 25 described in the first and second exemplary embodiments detects an angle between the arm 2 and the ground. In the flowchart in FIG. 9, in step S6, the opening and closing operation of the arm 2 is allowed within angles at which the X-ray tube 1 or the arm 2 does not interfere with the carriage portion 5.

After the image capturing, when the X-ray tube 1 is stored in the apparatus, control of operation opposite to the above-described operation may be performed to contract the arm 2 and to control the rotational angle of the support post 3 depending on the opening and closing angle of the arm 2. Further, a height detection sensor may be provided to the arm 2 to control release of the arm opening and closing lock unit depending on a height of the arm 2.

With the above-described structure, in setting the X-ray tube in photographing, or in storing the X-ray tube after the photographing, the X-ray tube and the arm are prevented from interfering with the carriage portion. Thus, the possibility that the operator's fingers are caught or the carriage portion having the control unit is damaged may be reduced. With the above-described structure, the mobile X-ray imaging apparatuses increased in the safety of operators and the apparatuses can be provided.

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (e.g., computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-223563 filed Oct. 5, 2012, which is hereby incorporated by reference herein in its entirety.

What is claimed is:

1. A mobile X-ray imaging apparatus comprising:
an X-ray tube configured to emit X-rays;
an extendable arm configured to support the X-ray tube;
a support post configured to move the extendable arm in a direction perpendicular to the ground;
a carriage portion configured to control the X-ray tube to emit the X-rays and to support the support post, and including a movable unit;
a monitor disposed on an upper surface of the carriage portion; and
an arm opening and closing unit configured to open and close the extendable arm with respect to the support post.

2. The mobile X-ray imaging apparatus according to claim 1, further comprising a support post extension and contraction unit configured to extend and contract the support post in the vertical direction.

3. The mobile X-ray imaging apparatus according to claim 1, wherein the extendable arm includes an arm extension and contraction lock unit configured to restrict the extending and contracting operation of the extendable arm, and a sensor configured to detect an opening and closing angle of the extendable arm, and
wherein, as long as the extendable arm is not set to a horizontal angle, the extension and contraction lock by the arm extension and contraction lock unit is not released.

4. The mobile X-ray imaging apparatus according to claim 1, wherein the arm opening and closing unit includes an arm opening and closing lock unit configured to fix an opening and closing angle of the extendable arm.

5. The mobile X-ray imaging apparatus according to claim 4, wherein the support post includes a support post rotation unit configured to rotate the support post around the vertical axis on the carriage portion, and a support post rotation detection unit configured to detect a rotational angle of the support post, and
wherein, within a range of rotational angles of the support post, the arm opening and closing lock unit of the extendable arm is not released.
